(19) United States
(12) Patent Application Publication
(10) Pub. No.: US 2021/0379082 A1
(43) Pub. Date: Dec. 9, 2021

(54) RECTAL IRRITATION CREAM WITH CBD OIL

(71) Applicant: Robert Jeffrey Sikes, Valdosta, GA (US)

(72) Inventor: Robert Jeffrey Sikes, Valdosta, GA (US)

(21) Appl. No.: 16/893,732

(22) Filed: Jun. 5, 2020

(51) Int. Cl. A61K 31/573; A61K 9/00; A61K 9/06; A61K 31/05; A61K 31/045; A61K 31/167

(52) U.S. Cl. CPC A61K 31/573; A61K 9/0031; A61K 31/167; A61K 31/05; A61K 31/045; A61K 9/06

(57) ABSTRACT

A rectal cream for increased rectal comfort that includes a lipophilic hydrophobic counter irritant, a lipophilic hydrophobic anesthetic, a lipophilic hydrophobic anti-inflammatory, and CBD oil. For example, the rectal cream includes menthol USP 0.5%, lidocaine USP, 2%, hydrocortisone USP 1%, and CBD Oil. In addition to providing immediate relief from rectal irritation, the rectal cream also promotes healing of the rectal mucosa and peri-anal skin, without the need to make dietary changes.

### FIELD OF THE INVENTION

This invention relates generally to rectal creams, and particularly to rectal creams for increased rectal comfort.

### BACKGROUND OF THE INVENTION

Everyone at some time suffers from rectal irritation and/or rectal itching. Whether from post-diarrheal episodes, improper cleaning after defecation, poor dietary intake, using rough toilet tissue, or from medically related issues such as Crohn's Disease or Irritable Bowel Syndrome, rectal irritation is a common problem.

Bile acids, which function as a surfactant, allow the digestion of dietary fats and oils. However, excessive secretion of bile acids, or improper reabsorption, results in an especially irritating situation for the rectal mucosa and surrounding peri-anal skin tissue. The rectum and peri-anal area can become irritated because bile acids act as surfactants or detergents, and can consequentially be toxic to cells.

Known remedies to protect the rectal mucosa and peri-anal skin include: application of skin barriers such as zinc oxide or Vaseline, Burrow's Solution (5% Aluminum Subacetate), 1% hydrocortisone cream, and oral antihistamines. Other strategies involved avoidance of irritants such as bubble baths or genital deodorants, dietary changes such as cutting back or eliminating coffee, colas, alcohol, citrus fruits, chocolate, spicy foods, and laxatives. Adding fiber to diet has also been suggested. Another suggestion is to avoid scratching the irritated/itching area, since scratching may further aggravate the problem.

The previous suggested remedies and strategies have been inadequate, partly because they fail to provide immediate relief from this irritating problem, and because they do not adequately address skin irritation and skin healing.

### SUMMARY OF THE INVENTION

The preparation of the invention provides immediate relief, substantially reduces skin irritation, and promotes skin healing.

Within 15 seconds of application, the user begins to feel a cooling effect of the irritated rectal area from the fast-acting counter-irritant ingredient, such as menthol USP, or Peppermint Oil.

The anti-inflammatory ingredient hydrocortisone USP, and the anesthetic ingredient lidocaine USP, take a few more minutes to take effect.

The need to scratch is quickly reduced because the ingredients synergistically provide support for palliative relief.

Finally, full spectrum CBD oil, which includes cannabidiol, supports skin healing and provides pain relief to the peri-anal area.

A general aspect of the invention is a rectal cream for increased rectal comfort. The rectal cream includes: a lipophilic hydrophobic counter irritant; a lipophilic hydrophobic anesthetic; a lipophilic hydrophobic anti-inflammatory; and CBD oil.

In some embodiments, the lipophilic hydrophobic counter irritant includes menthol.

In some embodiments, the lipophilic hydrophobic counter irritant is menthol USP 0.5%.

In some embodiments, the lipophilic hydrophobic anesthetic includes lidocaine.

In some embodiments, lipophilic hydrophobic anesthetic is lidocaine USP, 2%.

In some embodiments, the lipophilic hydrophobic anti-inflammatory includes hydrocortisone.

In some embodiments, the lipophilic hydrophobic anti-inflammatory is hydrocortisone USP 1%.

In some embodiments, the hydrocortisone USP 1% is not a chloride salt.

In some embodiments, the hydrocortisone USP is non-prescription strength.

Another general aspect of the invention is a rectal cream for increased rectal comfort that includes: a lipophilic hydrophobic counter irritant including menthol; a lipophilic hydrophobic anesthetic including lidocaine; a lipophilic hydrophobic anti-inflammatory including hydrocortisone; and CBD oil.

In some embodiments, the menthol is menthol USP 0.5%.

In some embodiments, the lidocaine is lidocaine USP, 2%.

In some embodiments, the hydrocortisone is hydrocortisone USP 1%.

In some embodiments, the hydrocortisone is non-prescription strength.

In some embodiments, the hydrocortisone is not a chloride salt.

Yet another general aspect of the invention is a rectal cream for increased rectal comfort that includes: menthol USP 0.5%; lidocaine USP, 2%; hydrocortisone USP 1%; and CBD Oil.

In some embodiments, the hydrocortisone USP 1% is not a chloride salt.

### BRIEF DESCRIPTION OF THE DRAWINGS

The invention will be more fully understood from the following Detailed Description, in conjunction with the following figures, wherein:

FIG. 1 is a process flow diagram showing a method for making an embodiment of the Rectal Irritation Cream With CBD Oil.

### DETAILED DESCRIPTION

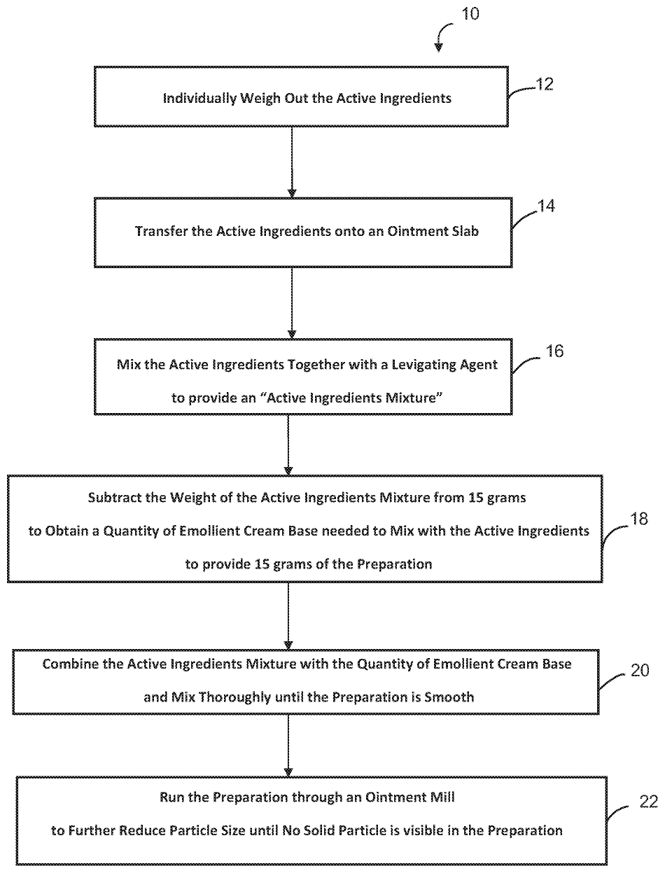

With reference to FIG. 1, a method 10 for making an embodiment of the Rectal Irritation Cream With CBD Oil is presented.

To prepare fifteen grams of the preparation, individually weigh out 12 the Active Ingredients. For example, using weigh boats and an analytical balance to individually weigh out the active ingredients:

| | |
|---|---|
| menthol USP | 75 mg |
| lidocaine USP | 300 mg |
| hydrocortisone USP | 150 mg |
| cannabidiol | 37.5 mg |

Assuming that a 15 gm quantity of the preparation is to be made including the given percentages of active ingredients, the corresponding weight percentages are:

| | |
|---|---|
| menthol USP | 0.5% |
| lidocaine USP | 2.0% |
| hydrocortisone USP | 1.0% |
| cannabidiol | 0.25% |

Next, transfer 14 the Active Ingredients onto an Ointment Slab.

Then, mix 16 the Active Ingredients together with a Levigating Agent to provide an “Active Ingredients Mixture”. For example, as a levigating agent use a small amount (e.g., 1-3 ml) of propylene glycol (or coconut oil or almond oil). Levigate thoroughly using a spatula, for example, until there is a smooth preparation to provide an “Active Ingredients Mixture”. Levigating the active ingredients means to reduce the particle size of the active ingredients to a small enough particle size so as to make a substantially homogeneous mixture.

Next, subtract 18 the weight of the Active Ingredients Mixture from fifteen grams to Obtain a Quantity of Emollient Cream Base needed to Incorporate the Active Ingredients to provide fifteen grams of the Preparation.

The Quantity of Emollient Cream Base made in the example below is 100 grams. Each ingredient is expressed as a percentage of the total of 100 grams by weight. The amounts in this example can be scaled so as to prepare only the Quantity of Emollient Cream Base needed to mix with the Active Ingredients so as to provide fifteen grams of the Preparation.

1. Phase a Ingredients to be Placed in Glass Beaker:

| | | |
|---|---|---|
| Coconut Oil | 4% | 4 grams |
| Cetearyl Alcohol | 2.5% | 2.5 grams |
| Sorbitan Stearate | 1.5% | 1.5 grams |
| Polysorbate 60 | 2.5% | 2.5 grams |
| Stearic Acid | 2% | 2 grams |
| Stearyl Palmitate | 3% | 3 grams |
| | 15.5% | 15.5 grams |

Heat Phase A to 160 degrees F.

2. Phase B Ingredients to be Placed in Glass Beaker:

| | | |
|---|---|---|
| Distilled Water | 78% | 78 grams (ml) |
| Propanediol 1,3 | 2% | 2 grams |
| Sorbitol | 0.2% | 0.2 grams |
| Sodium Gluconate | 0.2% | 0.2 grams |
| Blend Mixture until dissolved | | |
| Sprinkle in Carbomer | 0.5% | 0.5 grams |
| | 80.9% | 80.9 grams |

Blend well! Use a sheer mixer, homogenizer, or stick blender.

Heat Phase B to 160 degrees F.

Combine Melted Mixtures once all ingredients are melted by adding Phase B to Phase A. Blend well. Measure pH of the resultant mixtures. Should be less than 5

3. Phase C Mix in One-by-One to Mixture of A and B Above:

| | | |
|---|---|---|
| Silicone Gel (with blender) | 2.5% | 2.5 grams |
| Phenoxyethanol | 1% | 1 gram |
| May stir into mixture with small spatula | | |
| | 3.5% | 3.5 grams |

Adjust pH of resultant mixture to approximately 5.5 by adding

| | |
|---|---|
| Triethanolamine (TEA) | 1-2 drops. |

Blend well before measuring pH again. Repeat with 1-2 drops of TEA if necessary until pH is approximately 5.5. Allow to sit and cool. This will result in 100 grams of Emollient Cream Base.

Other examples of ingredients for making a suitable emollient cream base include:
1. Purified water, mineral oil, petrolatum, cetostearyl alcohol, propylene glycol, sodium lauryl sulfate, isopropyl palmitate, imidazolidinyl urea, methylparaben and propylparaben.
2. Water, petrolatum, cetyl alcohol, stearyl alcohol, mineral oil, polysorbate 80, BHT (butylated hydroxytoluene), disodium EDTA, methylchloroisothiazolinone, and methylisothiazolinone

One could also add Sheabutter, Macadamia Nut Oil, Cocoa Butter, Vitamin E Tocopherol, Fragrance, and/or Coconut Oil.

Next, combine 20 the Active Ingredients Mixture with the Quantity of Emollient Cream Base, and Mix Thoroughly until the Preparation is Smooth. For example, geometric dilution can be used to perform this mixing. Geometric dilution, also called geometric incorporation, is the process by which a homogeneous mixture of even distribution of two or more substances is achieved. When using this mixing method, the smallest quantity of active ingredient is mixed thoroughly with a proportion quantity of the diluent or base on the ointment slab. More diluent (base) is added in amounts proportionate to the volume of the mixture on the ointment slab. This process is repeated until all of the ingredients are incorporated in the mixture.

Finally, run 22 the Preparation through an Ointment Mill to further Reduce Particle Size until no solid particle is visible in the Preparation. For example, it may take one or two runs through the ointment mill such that no solid particle is visible. Using an ointment mill can result in better absorption for treating an irritated peri-anal area.

Other modifications and implementations will occur to those skilled in the art without departing from the spirit and the scope of the invention as claimed. Accordingly, the above description is not intended to limit the invention, except as indicated in the following claims.

What is claimed is:

1. A rectal cream for increased rectal comfort, the rectal cream comprising:
a lipophilic hydrophobic counter irritant;
a lipophilic hydrophobic anesthetic;
a lipophilic hydrophobic anti-inflammatory; and
CBD oil.

2. The rectal cream of claim 1, wherein the lipophilic hydrophobic counter irritant includes menthol.

3. The rectal cream of claim 1, wherein the lipophilic hydrophobic counter irritant is menthol USP 0.5%.

4. The rectal cream of claim 1, wherein the lipophilic hydrophobic anesthetic includes lidocaine.

5. The rectal cream of claim 1, wherein lipophilic hydrophobic anesthetic is lidocaine USP, 2%.

6. The rectal cream of claim 1, wherein the lipophilic hydrophobic anti-inflammatory includes hydrocortisone.

7. The rectal cream of claim 1, wherein the lipophilic hydrophobic anti-inflammatory is hydrocortisone USP 1%.

8. The rectal cream of claim 7, wherein the hydrocortisone USP 1% is not a chloride salt.

9. The rectal cream of claim 7, wherein the hydrocortisone USP is non-prescription strength.

10. A rectal cream for increased rectal comfort, the rectal cream comprising:
a lipophilic hydrophobic counter irritant including menthol;
a lipophilic hydrophobic anesthetic including lidocaine;
a lipophilic hydrophobic anti-inflammatory including hydrocortisone; and
CBD oil.

12. The rectal cream of claim 10, wherein the menthol is menthol USP 0.5%.

13. The rectal cream of claim 10, wherein the lidocaine is lidocaine USP, 2%.

14. The rectal cream of claim 10, wherein the hydrocortisone is hydrocortisone USP 1%.

16. The rectal cream of claim 10, wherein the hydrocortisone is non-prescription strength.

15. The rectal cream of claim 10, wherein the hydrocortisone is not a chloride salt.

16. A rectal cream for increased rectal comfort, the rectal cream comprising:
menthol USP 0.5%;
lidocaine USP, 2%;
hydrocortisone USP 1%; and
CBD Oil.

17. The rectal cream of claim 16, wherein the hydrocortisone USP 1% is not a chloride salt.
